(19) United States
(12) Patent Application Publication
(10) Pub. No.: US 2026/0138126 A1
(43) Pub. Date: May 21, 2026

(54) CATALYST FOR PRODUCING LACTIC ACID BY DEHYDROGENATION OF GLYCEROL AND MANUFACTURING METHOD THEREOF MANUFACTURING METHOD THEREOF

(71) Applicant: KOREA RESEARCH INSTITUTE OF CHEMICAL TECHNOLOGY, Daejeon (KR)

(72) Inventors: Young Kyu HWANG, Daejeon (KR); Dong Won HWANG, Daejeon (KR); In Hyoup SONG, Daejeon (KR); Ji Hoon KIM, Daejeon (KR); Ju Won MIN, Daejeon (KR); Anil Haribhau VALEKAR, Daejeon (KR); Macchindra Gulabrao CHANDGUDE, Daejeon (KR)

(73) Assignee: KOREA RESEARCH INSTITUTE OF CHEMICAL TECHNOLOGY, Daejeon (KR)

(21) Appl. No.: 19/449,004

(22) Filed: Jan. 14, 2026

Related U.S. Application Data
(63) Continuation of application No. PCT/KR2024/013115, filed on Sep. 2, 2024.

(30) Foreign Application Priority Data
Sep. 5, 2023 (KR) 10-2023-0117869

(51) Int. Cl. B01J 37/10; B01J 23/10; B01J 23/63; B01J 35/30; B01J 35/61; B01J 37/04; B01J 37/18; C07C 51/295

(52) U.S. Cl. CPC B01J 37/10; B01J 23/10; B01J 23/63; B01J 35/394; B01J 35/613; B01J 37/04; B01J 37/18; C07C 51/295

(57) ABSTRACT

The present invention relates to a catalyst for dehydrogenation of glycerol and to a catalyst for dehydrogenation of glycerol, which includes a group 10 element on the periodic table as a catalytically active component and a composite metal oxide composed of zirconium (Zr) and cerium (Ce) as a catalyst support, and a manufacturing method thereof.

### CROSS REFERENCE TO RELATED APPLICATION

The present application is a continuation of PCT International Application No. PCT/KR2024/013115, which has an International filing date of Sep. 2, 2024, and which claims priority to Korean Patent Application No. 10-2023-0117869, filed on Sep. 5, 2023, the entire contents of each of which is incorporated herein for all purposes by this reference.

### TECHNICAL FIELD

The present disclosure relates generally to a catalyst for producing lactic acid by dehydrogenation of glycerol and a manufacturing method thereof. More particularly, the present disclosure relates to a catalyst capable of effectively dehydrogenating glycerol under basic conditions to produce lactic acid with high efficiency, and to a manufacturing method thereof.

### BACKGROUND ART

In response to global issues such as resource depletion, global warming, and environmental pollution caused by the rapid consumption of fossil fuels, the development of new alternative energy sources for the future has emerged as a key challenge. Among these alternatives, hydrogen has gained increasing attention as a clean and pollution-free energy carrier and has become an essential next-generation energy source for applications such as future fuel cell vehicles using hydrogen fuel cells, as well as residential and industrial power generation.

Currently commercialized hydrogen production technologies predominantly rely on catalytic reforming reactions of natural gas or hydrogen by-products resulting from the dehydrogenation of petrochemical feedstocks. However, the future of the industry is expected to shift towards high value-added hydrogen production technologies utilizing alternative feedstocks. In particular, hydrogen production from biomass is considered an eco-friendly and future-oriented value-added technology due to its environmental benefits. For example, with the recent surge in global biodiesel production and consumption, the continuous accumulation of low-grade glycerol by-products (approximately 10% of the raw materials) has led to a decline in market prices due to oversupply and limiting improvements in the overall competitiveness of biodiesel.

Accordingly, converting such low value-added glycerol by-products into hydrogen, which is a high value-added clean fuel, has emerged as a major research topic in biomass-based hydrogen production, and extensive research and development efforts have been continuously undertaken in this area.

The glycerol is a promising hydrogen source because its dehydrogenation yields both hydrogen and high value-added lactic acid (LA). Consequently, over the past several decades, various dehydrogenation methods have been developed to produce hydrogen and lactate salts from glycerol dehydrogenation. Here, because the glycerol dehydrogenation reaction is typically carried out under basic conditions in the presence of metal hydroxide salts, there is an urgent demand for catalysts that exhibit high dehydrogenation activity even in strongly alkaline environments.

Meanwhile, as related art in the same technical field as the present disclosure, U.S. Patent Application Publication No. US 2015-0299082 A1 discloses a method for simultaneous production of lactic acid and propylene glycol from glycerol, in which glycerol is dehydrogenated using a copper (Cu)-containing dehydrogenation catalyst to produce lactate. Additionally, U.S. Patent Application Publication No. US 2012-0253067 A1 discloses a dehydrogenation catalyst for producing lactic acid from glycerol, including metals such as copper, platinum, ruthenium, palladium, and rhodium.

Although the aforementioned related art documents disclose technologies related to catalysts for converting glycerol into lactate via dehydrogenation, these catalysts exhibit insufficient stability under basic conditions and require further improvement in terms of catalytic activity.

### DOCUMENTS OF RELATED ART

### Patent Documents

(Patent Document 1) U.S. Patent Application Publication No. US 2015-0299082 A1 (published on Oct. 22, 2015)
(Patent Document 2) U.S. Patent Application Publication No. US 2012-0253067 A1 (published on Oct. 4, 2012)

### DISCLOSURE

### Technical Problem

Accordingly, the present disclosure has been made keeping in mind the above problems occurring in the related art, and an objective of the present disclosure is to provide a catalyst capable of effectively dehydrogenating glycerol under basic conditions, and a manufacturing method thereof.

### Technical Solution

In order to accomplish the above objective, the present disclosure provides a catalyst for dehydrogenation of glycerol, wherein the catalyst may include a composite metal oxide containing zirconium (Zr) and cerium (Ce) as a catalyst support, and wherein at least one active metal selected from Group 10 of the Periodic Table of Elements may be supported on the composite metal oxide.

In an embodiment of the present disclosure, the cerium (Ce) may be present in a content of 0.1 to 20 mol % based on a total molar content of the cerium (Ce) and the zirconium (Zr) in the composite metal oxide, and the active metal may be present in a content of 0.1 to 20 wt % based on a total weight of the catalyst.

Additionally, the present disclosure provides a manufacturing method of a catalyst for dehydrogenation of glycerol, the manufacturing method including: (i) mixing an active metal precursor with a dispersion solution in which a cerium-zirconium composite metal oxide support is dispersed, by dropwise adding an active metal precursor solution to the dispersion solution; (ii) an aging step of stirring the mixed solution for a predetermined period of time after step (i); (iii) a separation step of separating the cerium-zirconium composite metal oxide support on which the active metal precursor is supported from the mixed solution after step (ii); and (iv) calcining the cerium-zirconium composite metal oxide support on which the active metal precursor is supported after the separation step.

In an embodiment of the present disclosure, the manufacturing method may further include: prior to step (iv), drying the cerium-zirconium composite metal oxide support on which the active metal precursor is supported.

Additionally, the calcination in step (iv) may be carried out at a temperature of 400° C. to 800° C., and the manufacturing method may further include: after step (iv), reducing the calcined product at a temperature of 200° C. to 300° C.

The cerium-zirconium composite metal oxide support may be prepared by: (a) mixing a cerium source and a zirconium source; and (b) calcining the mixture.

Additionally, the present disclosure provides a method of dehydrogenating glycerol, the method including: bringing a solution in which glycerol is mixed with a hydroxide salt of an alkali metal or an alkaline earth metal into contact with a catalyst in which at least one element selected from Group 10 of the Periodic Table of Elements is supported on a composite metal oxide support composed of cerium and zirconium.

In the method of dehydrogenating glycerol, the hydroxide salt of the alkali metal or the alkaline earth metal may be used in a content of equal to or greater than 1.0 equivalent based on a molar content of the glycerol, and the cerium (Ce) may be present in a content of 0.1 to 20 mol % based on a total molar content of the cerium (Ce) and the zirconium (Zr) in the composite metal oxide support.

### Advantageous Effects

A catalyst for dehydrogenation of glycerol according to the present disclosure includes, as a catalytically active component, platinum (Pt) or the like, and employing, as a catalyst support, a composite metal oxide containing cerium (Ce) and zirconium (Zr). With this configuration, the disclosure provides the effect of enabling glycerol to be subjected to a dehydrogenation reaction under basic conditions.

### DESCRIPTION OF DRAWINGS

FIG. 1 illustrates the reaction mechanism of glycerol dehydrogenation.

FIG. 2 illustrates the results of transmission electron microscopy (TEM), particle size distribution, and energy-dispersive X-ray spectroscopy (EDS) analyses for catalysts manufactured in Production Example 1 (a), Production Example 2 (b), and Production Example 6 (c).

FIG. 3 illustrates s the results of catalyst reusability tests for Example 4 and Comparative Example 6.

### BEST MODE

Hereinbelow, a catalyst for dehydrogenation of glycerol and a manufacturing method thereof will be described in detail with reference to the accompanying drawings such that the disclosure can be easily embodied by one of ordinary skill in the art to which this disclosure belongs.

In the following description of the present disclosure, detailed descriptions of well-known functions or elements incorporated herein will be omitted to avoid obscuring the subject matter of the present disclosure.

Further, specific structural and functional descriptions of embodiments of the present disclosure disclosed herein are only for illustrative purposes of the preferred embodiments of the present disclosure, and do not encompass all of the technical spirit of the present disclosure. Accordingly, it should be understood that other equivalents and modifications could be made thereto without departing from the scope of the disclosure.

The present disclosure relates generally to a catalyst for dehydrogenation of glycerol and a manufacturing method thereof. More particularly, the present disclosure relates to a dehydrogenation catalyst and a manufacturing method thereof, which are used in a process in which glycerol is subjected to a dehydrogenation reaction by introducing water and a metal hydroxide salt into glycerol, thereby producing an organic acid metal salt in a dehydrogenated form of glycerol and hydrogen.

Hereinafter, the catalyst for dehydrogenation of glycerol and the manufacturing method thereof according to the present disclosure will be described in detail.

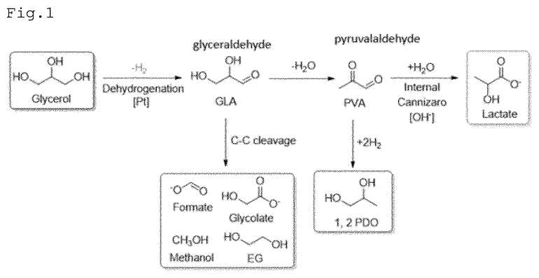

FIG. 1 illustrates the reaction mechanism of glycerol dehydrogenation. As illustrated in FIG. 1, glycerol is converted into glyceraldehyde through a dehydrogenation reaction, followed by conversion into pyruvalaldehyde, and ultimately yields lactic acid. Among these steps, the dehydrogenation of glycerol to glyceraldehyde is a key step in the overall reaction. It has been reported that, in order to convert glycerol into glyceraldehyde via a glyceroxide ion intermediate, a high temperature (exceeding 550 K) is required in a hydrothermal process. However, at such high temperatures, side reactions driven by the decomposition of pyruvalaldehyde or lactic acid proceed to a significant extent, thereby resulting in a reduced final selectivity toward lactic acid. To mitigate this issue, conducting the reaction at a lower temperature under basic conditions may be considered. Nevertheless, under basic conditions, a problem arises in that catalyst stability is significantly reduced.

The catalyst for dehydrogenation of glycerol according to the present disclosure is advantageous in that it exhibits high stability under basic conditions while also providing high dehydrogenation efficiency of glycerol.

The catalyst of the present disclosure may include, as a catalytically active component, at least one element selected from Group 10 of the Periodic Table of Elements, and include, as a catalyst support, a composite metal oxide composed of cerium (Ce) and zirconium (Zr). That is, the catalyst for dehydrogenation of glycerol according to the present disclosure may include a cerium-zirconium composite metal oxide (CeₓZr₁₋ₓO₂) on which at least one Group 10 element, such as platinum (Pt) and palladium (Pd), is supported as an active metal.

When cerium and zirconium are present in the form of such a composite metal oxide, the catalyst may exhibit excellent stability even under basic conditions, and both the glycerol conversion rate and the selectivity toward lactic acid may be significantly improved.

In the catalyst of the present disclosure, the content of the active metal is preferably within a range of equal to or less than 20 wt % based on the total weight of the catalyst, and more preferably within a range of 0.1 to 10 wt %. When the content of the active metal is equal to or greater than 0.1 wt %, the conversion of glycerol increases. However, when the content of the active metal exceeds 20 wt %, C-C bond cleavage is promoted due to the increased content of the active metal, resulting in decreased selectivity. Accordingly, the increase in catalytic activity relative to the increase in active metal content is not significant, which may be economically unfavorable.

Additionally, in the catalyst for dehydrogenation of glycerol according to the present disclosure, the conversion of glycerol under basic conditions varies depending on the mole ratio of cerium (Ce) to zirconium (Zr) in the composite metal oxide serving as the catalyst support. In this regard, the catalyst for dehydrogenation of glycerol according to the present disclosure preferably has a cerium (Ce) content of 0.1 to 20 mol %, and more preferably 1 to 10 mol %, based on the total molar content of cerium (Ce) and zirconium (Zr) in the catalyst support.

When the cerium content is within the range of 0.1 to 20 mol % relative to the total molar content of cerium and zirconium, the conversion of glycerol and the stability under basic conditions may be significantly improved. However, when the cerium content exceeds 20 mol %, a phase transition of ZrO₂ from a monoclinic structure to a tetragonal structure may occur, resulting in a reduction in catalytic activity.

A method for preparing a composite metal oxide catalyst support composed of cerium and zirconium according to the present disclosure may include: (a) mixing a cerium source and a zirconium source; and (b) calcining the mixture.

Step (a) is a step of forming a cerium-zirconium composite by mixing the cerium source and the zirconium source. The cerium source and the zirconium source are not particularly limited as long as they contain cerium atoms and zirconium atoms, respectively. For example, the cerium source and the zirconium source may include salts, complexes, or the like of cerium and zirconium, and specifically, may be at least one selected from nitrates, sulfates, phosphates, halide salts, alkoxide salts, oxynitrates, hydroxide salts, acetate salts, alkyl salts, and hydrates thereof.

The mixing ratio of the zirconium source and the cerium source may be such that cerium is used in a content of 0.1 to 20 mol %, based on the total molar content of zirconium and cerium, as described above.

Meanwhile, the mixing of the cerium source and the zirconium source is not particularly limited as long as it is a method capable of forming a composite of cerium and zirconium. For example, the mixing method may include a hydrothermal synthesis method, a co-precipitation method, an impregnation method, a mechanical mixing method, a deposition method, or the like, and preferably includes a hydrothermal synthesis method or a co-precipitation method.

In an embodiment, among the aforementioned mixing methods, the hydrothermal synthesis method may involve adding a base to the cerium source and the zirconium source to prepare a mixed solution of the cerium source, the zirconium source, and the base, followed by hydrothermal synthesis reaction. In this case, the base may be at least one selected from urea, aqueous ammonia, NaOH, organic amines, and the like, and preferably urea. The content of the base is not particularly limited, but it may be added such that the pH of the mixed solution containing the cerium precursor and the zirconium precursor is adjusted to equal to or higher than 8.

The hydrothermal synthesis may be carried out at a temperature of 100° C. to 250° C. for a predetermined period of time, for example, 10 to 30 hours. When the reaction temperature is lower than 100° C., the precursors may not react sufficiently due to the low reaction temperature, resulting in a low yield. On the other hand, when the reaction temperature exceeds 250° C., particle agglomeration or excessive particle growth may occur due to the high reaction temperature.

In another embodiment, among the aforementioned mixing methods, the co-precipitation method may involve adding a base to a cerium source and a zirconium source to prepare a mixed solution of the cerium source, the zirconium source, and the base, followed by co-precipitation reaction. In this case, the co-precipitation may be carried out at a temperature ranging from room temperature to equal to or lower than 100° C. for a predetermined period of time.

Meanwhile, step (b) is a step of obtaining a composite metal oxide by calcining the mixture obtained in step (a). Prior to the calcination, a step of pre-drying the mixture obtained in step (a) may be performed. In this case, the drying may be carried out using known methods and apparatuses such as a vacuum oven, hot air drying, constant temperature and humidity drying, and microwave drying. The drying may be performed at a temperature of 60° C. to 200° C. so as to achieve sufficient drying.

The calcination may be carried out in a temperature range of 350° C. to 800° C., typically for 2 to 8 hours. When the calcination temperature is lower than 350° C., the formation of the composite metal oxide may be incomplete due to insufficient calcination. On the other hand, when the calcination temperature exceeds 800° C., particle agglomeration or excessive particle growth may occur due to the high calcination temperature.

Thereafter, an active metal such as platinum (Pt) may be supported on the surface of the composite metal oxide obtained by the above-described method, thereby manufacturing a catalyst for dehydrogenation of glycerol. The supporting method is not particularly limited and may be any method commonly used in the art. For example, adsorption, evaporation drying, spraying, incipient wetness impregnation, or the like may be employed.

By way of example, the manufacturing method of the catalyst may include: (i) mixing an active metal precursor with a dispersion solution in which a cerium-zirconium composite metal oxide support is dispersed, by dropwise adding an active metal precursor solution to the dispersion solution; (ii) an aging step of stirring the mixed solution for a predetermined period of time after step (i); (iii) a separation step of separating the cerium-zirconium composite metal oxide support on which the active metal precursor is supported from the mixed solution after step (ii); and (iv) calcining the cerium-zirconium composite metal oxide support on which the active metal precursor is supported after the separation step.

In step (i), the active metal precursor may be least one selected from nitrates, sulfates, phosphates, halide salts, alkoxide salts, oxynitrates, hydroxide salts, acetate salts, alkyl salts, and hydrates thereof, which contains at least one element selected from Group 10 of the Periodic Table of Elements. Preferably, a halide salt hydrate may be used. Additionally, a solvent or dispersion medium used in the active metal precursor solution and in the dispersion solution in which the cerium-zirconium composite metal oxide support is dispersed may be the same as or different from each other. Any solvent or dispersion medium capable of dissolving the active metal precursor may be employed without particular limitation, but water is preferably used.

Step (ii) is a step in which, after the adding of the active metal precursor solution to the support dispersion solution is completed, the mixture is stirred for a predetermined period of time such that the active metal precursor is uniformly supported on the support. Step (ii) may be carried out at a temperature ranging from room temperature to a temperature equal to or lower than the boiling point of the solvent. The predetermined period of time may vary depending on conditions such as temperature, loading content, and the size and content of the support, and may be, for example, 4 to 8 hours.

Step (iii) is a step of separating the support on which the active metal precursor is supported from the solution. The separation may be carried out using conventional means, for example, filtration using a filter.

Step (iv) is a step of calcining the support on which the active metal precursor is supported. Prior to the calcination, a step of pre-drying the cerium-zirconium composite metal oxide support on which the active metal precursor is supported and obtained in step (iii) may be performed. In this case, the drying may be carried out using known methods and apparatuses such as a vacuum oven, hot air drying, constant temperature and humidity drying, and microwave drying. The drying may be performed at a temperature of 60° C. to 200° C. so as to achieve sufficient drying. The calcination may be carried out in a temperature range of 400° C. to 800° C., typically for 1 to 5 hours.

After the calcination, a step of reducing the calcined product may be performed. The reduction step may be carried out under a reducing atmosphere in a temperature range of 200° C. to 300° C.

The present disclosure also provides a method of dehydrogenating glycerol. Specifically, a dehydrogenation reaction of glycerol may be carried out by bringing a solution in which glycerol is mixed with a hydroxide salt of an alkali metal or an alkaline earth metal into contact with a catalyst in which at least one element selected from Group 10 of the Periodic Table of Elements is supported on a composite metal oxide composed of cerium and zirconium.

The hydroxide salt of the alkali metal or alkaline earth metal may be, for example, at least one selected from KOH, NaOH, LiOH, Ca(OH) 2, and Mg(OH) 2. The reaction temperature is preferably in a range of 150° C. to 300° C. The hydroxide salt of the alkali metal or alkaline earth metal may be used in a content of equal to or greater than about 1.0 equivalent, and preferably 1.0 equivalent to 3.0 equivalents, based on the molar content of glycerol used. Additionally, prior to the reaction, the air inside a reactor may be replaced with an inert gas.

Hereinafter, the catalytic activity of the catalyst for dehydrogenation of glycerol according to the present disclosure will be examined through Examples. It should be noted that the following Examples are provided to illustrate one or more preferred embodiments of the present disclosure, and the disclosure is not limited to these embodiments. Various modifications may be made to the following Examples without departing from the scope of the present disclosure.

### <Catalyst Manufacture>

Production Example 1: Manufacture of 2 wt % Pt/5Ce—ZrO₂ Catalyst

### 1-1: Preparation of Support

A mixed solution was prepared by dissolving 18.5 g of ZrOCl₂·8H₂O, 1.3 g of Ce(NO₃)₃·6H₂O, and 36 g of urea in 140 ml of distilled water. The prepared mixed solution was transferred into a 250 mL Teflon-lined autoclave, heated to 200° C., and maintained at this temperature for 20 hours to carry out a hydrothermal synthesis reaction. After completion of the reaction, the resulting solid was collected by filtration, washed several times with deionized water, and then dried in a drying oven at 100° C. for 12 hours to obtain a dried product. The dried product was placed in a calcination furnace through which air was supplied at a flow rate of 150 cc/min, and the temperature of the furnace was increased to 400° C. at a rate of 5° C./min. The dried product was then calcined at the same temperature for 4 hours to yield a 5Ce—ZrO₂ composite metal oxide. Here, the numeral “5” in 5Ce—ZrO₂ indicates that the Ce content corresponds to 5 mol % relative to the total molar content of Ce and Zr.

1-2: Manufacture of Catalyst Supported with Active Metal

The composite metal oxide obtained in Production Example 1-1 was added to a vessel containing distilled water, and a solution in which H₂PtCl₆·6H₂O was dissolved in distilled water was dropwise added thereto. The resulting mixture was stirred at room temperature for 6 hours. After stirring, the solid was collected by filtration and dried overnight in a drying oven at 100° C. to obtain a dried product. The dried product was placed in a calcination furnace through which air was supplied at a flow rate of 150 cc/min, and the temperature of the furnace was increased to 500° C. at a rate of 5° C./min. The dried product was then calcined at the same temperature for 2 hours. Thereafter, prior to carrying out a dehydrogenation reaction in a reactor, the calcined product was subjected to a reduction reaction at 250° C. for 2 hours while flowing a mixed gas of H₂/Ar containing 10% hydrogen at a flow rate of 150 cc/min, thereby yielding a final 2 wt % Pt/5Ce—ZrO₂ catalyst. The Pt content in the catalyst was 2 wt % based on the total weight of the catalyst.

### Production Example 2: Manufacture of 2 wt % Pt/10Ce—ZrO₂ Catalyst

Except that, during the support preparation process, the amount of ZrOCl₂·8H₂O was adjusted such that the Ce content corresponded to 10 mol % relative to the total molar content of Ce and Zr, the same procedure as that of Production Example 1 was carried out to yield a Pt/10Ce—ZrO₂ catalyst.

### Production Example 3: Manufacture of 2 wt % Pd/5Ce—ZrO₂ Catalyst

Except that K₂PdCl₄ was used instead of H₂PtCl₆·6H₂O during the catalyst manufacturing process, the same procedure as that of Production Example 1 was carried out to yield a 2 wt % Pd/5Ce—ZrO₂ catalyst.

### Production Example 4: Manufacture of 2 wt % Ru/5Ce—ZrO₂ Catalyst

Except that RuCl₃·3H₂O was used instead of H₂PtCl₆·6H₂O during the catalyst manufacturing process, the same procedure as that of Production Example 1 was carried out to yield a 2 wt % Ru/5Ce—ZrO₂ catalyst.

### Production Example 5: Manufacture of 2 wt % Ir/5Ce—ZrO₂ Catalyst

Except that IrCl₃·xH₂O was used instead of H₂PtCl₆·6H₂O during the catalyst manufacturing process, the same procedure as that of Production Example 1 was carried out to yield a 2 wt % Ir/5Ce—ZrO₂ catalyst.

### Production Example 6: Manufacture of 2 wt % Pt/ZrO₂ Catalyst

Except that a commercially available zirconia (Sigma-Aldrich) was used as a support without performing the support preparation process, the same procedure as that of Production Example 1 was carried out to yield a 2 wt % Pt/ZrO₂ catalyst.

### <Catalyst Characterization>

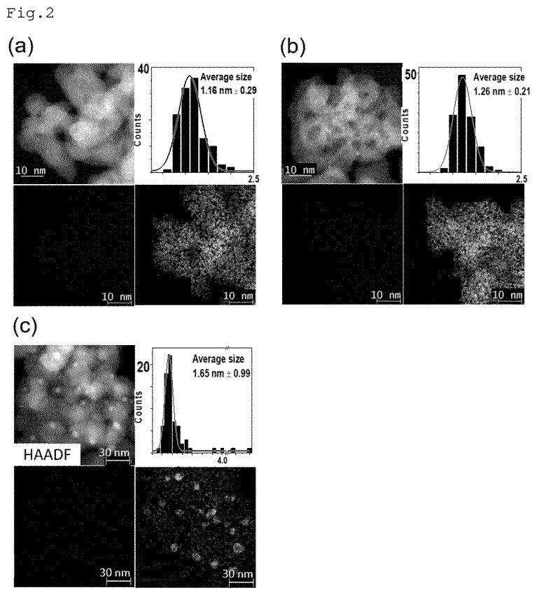

The catalysts manufactured in Production Examples 1, 2, and 6 were analyzed using transmission electron microscopy (TEM), energy-dispersive X-ray spectroscopy (EDS), and a particle size analyzer, and the results are shown in FIG. 2. In FIG. 2, (a) shows the results for the catalyst of Production Example 1, (b) shows the results for the catalyst of Production Example 2, and (c) shows the results for the catalyst of Production Example 6.

Referring to FIG. 2, in the case of the catalysts of Production Examples 1 and 2, which employed the composite metal oxide support composed of zirconium and cerium according to the present disclosure, platinum (Pt) was very uniformly distributed on the support. On the contrary, in the catalyst of Production Example 6, which used zirconia (ZrO₂) as the support without cerium, platinum (Pt) was present in a relatively aggregated form, and the diameter of platinum (Pt) particles was larger than those observed in the catalysts of Production Examples 1 and 2.

### <Glycerol Dehydrogenation Reaction Activity>

### EXAMPLE 1

23.02 g of glycerol (250 mmol), 24.04 g of KOH (375 mmol), 23 mL of distilled water, and 0.3 g of the catalyst manufactured in Production Example 1 were charged into a reactor. Nitrogen gas was purged into the reactor to completely replace the air inside the reactor. Thereafter, the reactor was heated to 200° C. and maintained at this temperature for 2 hours. After completion of the reaction, the reactor was cooled to room temperature. The reaction results were measured and are shown in Table 1.

### EXAMPLE 2

Except that the catalyst of Production Example 2 was used instead of the catalyst of Production Example 1, the reaction results were measured in the same manner as in Example 1, and the results are shown in Table 1.

### EXAMPLE 3

Except that the catalyst of Production Example 3 was used instead of the catalyst of Production Example 1, the reaction results were measured in the same manner as in Example 1, and the results are shown in Table 1.

### COMPARATIVE EXAMPLE 1

Except that neither the catalyst nor KOH was used, the reaction results were measured in the same manner as in Example 1, and the results are shown in Table 1.

### COMPARATIVE EXAMPLE 2

Except that the catalyst was not used, the reaction results were measured in the same manner as in Example 1, and the results are shown in Table 1.

### COMPARATIVE EXAMPLE 3

Except that the catalyst of Production Example 4 was used instead of the catalyst of Production Example 1, the reaction results were measured in the same manner as in Example 1, and the results are shown in Table 1.

### COMPARATIVE EXAMPLE 4

Except that the catalyst of Production Example 5 was used instead of the catalyst of Production Example 1, the reaction results were measured in the same manner as in Example 1, and the results are shown in Table 1.

### COMPARATIVE EXAMPLE 5

Except that the catalyst of Production Example 6 was used instead of the catalyst of Production Example 1, the reaction results were measured in the same manner as in Example 1, and the results are shown in Table 1.

In Table 1, SBET represents the specific surface area of catalysts; GLY represents glycerol; LA represents lactic acid; CONV. represents conversion; Yield represents yield; FA represents formic acid; 1,2PDO represents 1,2-propanediol; EG represents ethylene glycol; MA represents methanol; and GA represents glycolate.

TABLE 1

| | | | | | GLY | LA | FA | 1,2PDO | EG | MA | GA |
|---|---|---|---|---|---|---|---|---|---|---|---|
| | | S_{BET} | GLY | KOH | conv. | yield | yield | yield | yield | Yield | yield |
| Classification | Catalyst | (m²/g) | (g/mmol) | (g/mmol) | (%) | (%) | (%) | (%) | (%) | (%) | (%) |
| Comparative | — | — | 23/250 | — | 0 | 0 | 0 | 0 | 0.3 | 0 | 0 |
| Example 1 | | | | | | | | | | | |
| Comparative | — | — | 23/250 | 21/375 | 30.6 | 20.9 | 1.7 | 8.7 | 0.3 | 0.2 | 1.3 |
| Example 2 | | | | | | | | | | | |
| Example 1 | Production | 70.1 | 23/250 | 21/375 | 94.4 | 80.5 | 2.9 | 6.8 | 0.3 | 0.8 | 2.6 |
| | Example 1 | | | | | | | | | | |
| Example 2 | Production | 58.3 | 23/250 | 21/375 | 98.5 | 72.6 | 3.2 | 7.7 | 0.3 | 0.6 | 2.8 |
| | Example 2 | | | | | | | | | | |
| Example 3 | Production | 56.3 | 23/250 | 21/375 | 89.3 | 63.9 | 2.3 | 12.4 | 0.3 | 0.6 | 3.2 |
| | Example 3 | | | | | | | | | | |
| Comparative | Production | 57.0 | 23/250 | 21/375 | 40.5 | 33.5 | 1.8 | 4.0 | 0.2 | 0.2 | 1.8 |
| Example 3 | Example 4 | | | | | | | | | | |
| Comparative | Production | 58.2 | 23/250 | 21/375 | 36.0 | 20.3 | 1.1 | 6.5 | 0.1 | 0.1 | 0.8 |
| Example 4 | Example 5 | | | | | | | | | | |
| Comparative | Production | 25.2 | 23/250 | 21/375 | 83.4 | 69.5 | 1.7 | 5.8 | 0.3 | 0.6 | 2.0 |
| Example 5 | Example 6 | | | | | | | | | | |

### <Catalyst Stability>

### EXAMPLE 4

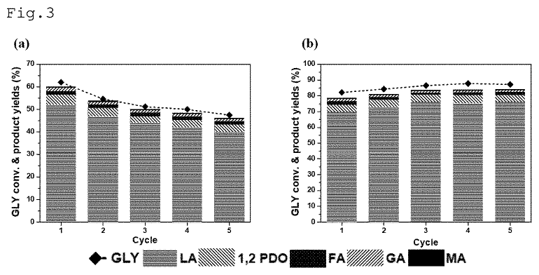

In Example 1, after completion of the reaction, the catalyst was recovered by filtration and reused as a catalyst to repeatedly carry out the glycerol dehydrogenation reaction in the same manner as in Example 1. The results thereof are shown in FIG. 3(a).

### COMPARATIVE EXAMPLE 6

In Comparative Example 1, after completion of the reaction, the catalyst was recovered by filtration and reused as a catalyst to repeatedly carry out the glycerol dehydrogenation reaction in the same manner as in Example 1. The results thereof are shown in FIG. 3(b).

As can be seen from FIG. 3, in the case of Example 4, the reaction activity did not decrease even after the catalyst was reused five times or more, and rather showed a tendency to slightly increase. On the contrary, in the case of Comparative Example 6, the reaction activity tended to decrease as the number of catalyst reuse cycles increased. These results demonstrate that the catalyst of the present disclosure exhibits excellent stability under basic conditions.

Although the present disclosure has been described with reference to examples, the examples are presented only for illustrative purposes. It will be appreciated by those skilled in the art that various modifications and equivalents may be made to the examples. Accordingly, the technical scope of technical protection of the present disclosure should be defined by the appended claims.

### INDUSTRIAL APPLICABILITY

The present disclosure relates to a catalyst for dehydrogenation of glycerol, the catalyst including, as a catalytically active component, platinum (Pt) or the like, and employing, as a catalyst support, a composite metal oxide containing cerium (Ce) and zirconium (Zr). With this configuration, glycerol can be stably subjected to a dehydrogenation reaction under basic conditions. Accordingly, the catalyst of the present disclosure can find effective application in industrial fields for producing not only hydrogen but also high value-added lactic acid (LA) from glycerol.

1. A catalyst for producing lactic acid by dehydrogenation of glycerol in the presence of a base,
wherein the catalyst comprises a composite metal oxide containing zirconium (Zr) and cerium (Ce) as a catalyst support, and wherein at least one active metal selected from Group 10 of the Periodic Table of Elements is supported on the composite metal oxide.

2. The catalyst of claim 1, wherein the cerium (Ce) is present in a content of 0.1 to 20 mol % based on a total molar content of the cerium (Ce) and the zirconium (Zr) in the composite metal oxide.

3. The catalyst of claim 1, wherein the active metal is present in a content of 0.1 to 20 wt % based on a total weight of the catalyst.

4. A manufacturing method of a catalyst for producing lactic acid from glycerol, the manufacturing method comprising:
(i) mixing an active metal precursor with a dispersion solution in which a cerium-zirconium composite metal oxide support is dispersed, by dropwise adding an active metal precursor solution to the dispersion solution;
(ii) an aging step of stirring the mixed solution for a predetermined period of time after step (i);
(iii) a separation step of separating the cerium-zirconium composite metal oxide support on which the active metal precursor is supported from the mixed solution after step (ii); and
(iv) calcining the cerium-zirconium composite metal oxide support on which the active metal precursor is supported after the separation step.

5. The manufacturing method of claim 4, further comprising: prior to step (iv), drying the cerium-zirconium composite metal oxide support on which the active metal precursor is supported.

6. The manufacturing method of claim 4, wherein the calcination in step (iv) is carried out at a temperature of 400° C. to 800° C.

7. The manufacturing method of claim 4, further comprising:
after step (iv), reducing the calcined product at a temperature of 200° C. to 300° C.

8. The manufacturing method of claim 4, wherein the cerium (Ce) is present in a content of 0.1 to 20 mol % based on a total molar content of the cerium (Ce) and the zirconium (Zr) in the cerium-zirconium composite metal oxide.

9. The manufacturing method of claim 4, wherein the cerium-zirconium composite metal oxide support is prepared by:
(a) mixing a cerium source and a zirconium source; and
(b) calcining the mixture.

10. A method of producing lactic acid from glycerol, the method comprising:
bringing a solution in which glycerol is mixed with a hydroxide salt of an alkali metal or an alkaline earth metal into contact with a catalyst in which at least one element selected from Group 10 of the Periodic Table of Elements is supported on a composite metal oxide support composed of cerium and zirconium.

11. The method of claim 10, wherein the hydroxide salt of the alkali metal or the alkaline earth metal is used in a content of equal to or greater than 1.0 equivalent based on a molar content of the glycerol.

12. The method of claim 10, wherein the cerium (Ce) is present in a content of 0.1 to 20 mol % based on a total molar content of the cerium (Ce) and the zirconium (Zr) in the composite metal oxide support.
